(19) United States
(12) Patent Application Publication
(10) Pub. No.: US 2007/0104666 A1
(43) Pub. Date: May 10, 2007

(54) Styptic formulation and method of use

(76) Inventor: Michael Haltom, Dana Point, CA (US)

Correspondence Address: PATENT LAW & VENTURE GROUP, 2424 S.E. BRISTOL, SUITE 300, NEWPORT BEACH, CA, 92660, US

(21) Appl. No.: 11/268,391

(22) Filed: Nov. 7, 2005

(51) Int. Cl. A61K 8/26

(52) U.S. Cl. 424/68

(57) ABSTRACT

An astringent formulation of a double sulfate of a trivalent metal in combination with a univalent metal, cornstarch, water and a fragrance as an astringent for shaving and a method of use.

### CROSS-REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION-BY-REFERENCE OF MATERIAL SUBMITTTED ON A COMPACT DISC

Not applicable.

### REFERENCE TO A “MICROFICHE APPENDIX”

Not applicable.

### BACKGROUND OF THE INVENTION

1. Field of the Present Disclosure

This disclosure relates generally to topical formulations such as astringents and styptics.

2. Description of Related Art including information disclosed under 37 CFR 1.97 and 1.98

The word “styptic” is taken from the Greek “stuptikos” meaning to contract. Styptic materials are commonly used in commercial products in gel, powder or solid form and with a variety of ingredients for contracting tissue or blood vessels, as for instance to stop bleeding. Such materials perform the functions of a hemostatic or astringent. In the commercial market, various styptic devices are known such as styptic pencils. As an example, Requa Manufacturing, Inc. provides a commercially available styptic pencil having aluminum sulfate as the active ingredient. Such a pencil is used by wetting the tip and applying it to a bleeding point on the skin surface. A liquid filled swab is commercially available as well which, when the swab is snapped, a liquid containing an astringent flows into a cotton tip for veterinary applications. Gels and powder styptic materials are also commercially available primarily for use on dogs, cats and birds.

Lozeau et al., U.S. Pat. No. 5,688,497, discloses shaving aids comprising at least one active ingredient which provides a physiological cooling effect and a coagulant. Goldstein et al., U.S. Pat. No. 4,963,350, discloses a stable soothing hydroalcoholic skin preparation for shaving purposes employing alcohol as a carrier or solvent for the remainder of the composition comprising a combined allantoin-boric acid hydrolyzed solution in combination with glycerine, menthol, ethyl alchohol and fragrance. Balazes et al., U.S. Pat. No. 5,266,563, discloses water based, viscoelastic compositions comprising a mixture of high molecular weights hyaluronic acid salts and water soluble poly(ethylene oxides) and cosmetic formulations. Grollier et al., U.S. Pat. No. 5,104,643, and U.S. Pat. No. 4,957,732, discloses a shaving composition for the skin based on polyorganosiloxanes containing a hydroxyalkyl group and process for use. The composition is intended for shaving the skin, and contains, in a cosmetically acceptable medium a foaming agent, a polyorganosiloxane containing a hydroxyl group, of formula I: ##STR1## in which the radicals R, which may be identical or different, denote methyl or phenyl, at least 60 mol % of the radicals R being methyl. Haws et al., U.S. Pat. No. 6,754,958, discloses a safety razor having a handle formed by a refillable container. After filling with water a tablet containing a carbon dioxide gas generating substance is added to the water and the gas pressure is used to deliver liquid from the container to the underside of the razor blade unit. The tablet also includes a substance which upon being mixed with water produces a fluid which may have desirable properties for application to the skin during shaving, such as lubrication, moisturizing, applying a fragrance, chemical treatment of hairs, bacterial or medicinal treatment of the skin and/or blood coagulation. The container can be equipped with a dispenser (30) operable to deliver tablets into the water filled container. Rosen et al., U.S. Pat. No. 6,703,009, discloses Topical compositions and methods are disclosed for the treatment of pseudofolliculitis barbae and ingrown hair. The compositions comprise acetylsalicylic acid, propylene glycol, glycerine, and isopropyl alcohol. The method involves the step of applying the composition to the beard areas of the face after shaving. The acetylsalicylic acid is dissolved in a solvent mixture comprising propylene glycol, glycerine, and isopropyl alcohol. The acetylsalicylic acid is present in the range of between about 5 percent by weight per unit volume of the solvent mixture up to saturation of the solvent mixture. The solvent mixture comprises propylene glycol in the range of about 5 to 15 percent by volume, glycerine in the range of about 1 to 10 percent by volume, and the balance of the volume made up with isopropyl alcohol alone or a solution comprising at least 50 percent by volume of isopropyl alcohol. The isopropyl alcohol can be in a solution with water, methanol or ethanol, provided that the polarity of the resulting solution is not so high that the acetylsalicylic acid would readily precipitate from the solution at ordinary room temperature ranges. Rico, U.S. Pat. No. 6,375,942, discloses a skin healing ointment including zinc oxide which promotes blood coagulation. Martin et al., US 20030077343, discloses a method for constricting blood vessels, inhibiting angiogenesis, and/or reducing non-inflammatory redness in the skin by the topical administration of a composition comprising a Feverfew extract. Filipski et al., US. 2003/0124083, discloses A soap-free shaving and moisturizing composition which maintains superior performance attributes while avoiding the harshness and drying associated with soap-based shave preparations is described. The shaving and moisturizing composition preferably comprises deionized water, a humectant, a water-based silicone lubricant, and sodium hyaluronate. The composition can also contain a conditioner, preservatives, allantoin, and various heavy alkaloid extracts. The present invention is a single shaving composition that performs as a shave cream, an after shave, and a moisturizer. Applying the composition does not require the application of water on the shave area or that water be added to the shaving composition, however water can be applied if desired. In addition, use of the shaving composition does not require a hydration wait time between applying the composition and shaving. Once shaving is complete, any residual shaving composition on the shaved area is rubbed into the skin and acts as an after shave and a long-term moisturizer. Shaving can be performed by using a multiple blade razor having an open flow design, thereby reducing resistance while shaving and decreasing dryness and irritation of the skin.

The related art described above discloses skin applied formulations primarily for use or in relation to the shaving of skin surfaces and particularly for stopping blood flow from a break in the skin. However, the prior art fails to disclose the use of the ingredients of the present formulation and in particular the proportions of the ingredients. The present disclosure distinguishes over the prior art providing heretofore unknown advantages as described in the following summary.

### BRIEF SUMMARY OF THE INVENTION

This disclosure teaches certain benefits in construction and use which give rise to the objectives described below.

The word “styptic” is taken from the Greek “stuptikos” meaning to contract. Styptic is commonly used to any of a variety of products in powder or solid form that are used for contracting tissue or blood vessels, as for instance to stop bleeding. Such materials perform the functions of a hemostatic or astringent. In the commercial market various styptic devices are known such as styptic pencils and powders. As an example, Requa Manufacturing, Inc. provides a commercial styptic pencil having aluminum sulfate as the active ingredient. Such a pencil is used by wetting the tip and applying it to a skin cut. In the powder form

A primary objective inherent in the above described apparatus and method of use is to provide advantages not taught by the prior art.

Another objective is to provide a topical formulation that has advantage of stopping the flow of blood from minor shaving cuts without pain while operating as a general skin surface salve.

A further objective is to provide such a topical that has the advantage of being able to be washed off the skin immediately without blood flow restarting.

Other features and advantages of the present invention will become apparent from the following more detailed description, taken in conjunction with the accompanying drawings, which illustrate, by way of example, the principles of the presently described apparatus and method of its use.

### BRIEF DESCRIPTION OF THE SEVERAL VIEWS OF THE DRAWING(S)

Illustrated in the accompanying drawing(s) is at least one of the best mode embodiments of the present invention In such drawing(s):

FIGS. 1-3 are perspective views of the use of the presently described formulation.

### DETAILED DESCRIPTION OF THE INVENTION

The above described drawing figures illustrate the described apparatus and its method of use in at least one of its preferred, best mode embodiment, which is further defined in detail in the following description. Those having ordinary skill in the art may be able to make alterations and modifications what is described herein without departing from its spirit and scope. Therefore, it must be understood that what is illustrated is set forth only for the purposes of example and that it should not be taken as a limitation in the scope of the present apparatus and method of use.

Described now in detail is an astringent formulation comprising a mixture of: a double sulfate of a trivalent metal in combination with a univalent metal; cornstarch; water and a fragrance. The double sulfate of a trivalent metal is preferably one of: aluminum, chromium and iron. The univalent metal is preferably one of: potassium and sodium. The double sulfate and univalent metal is preferably aluminum potassium sulfate, AlK(SO₄)₂. 12H₂O. Preferably, the mixture of aluminum potassium sulfate comprises between 60% and 70% of the mixture, the cornstarch comprises between 10% and 20% of the mixture, and the water and fragrance comprise the balance of the mixture. More particularly, the mixture of aluminum potassium sulfate, cornstarch, and water are in the approximate proportion of 65%, 15% and 20% respectively.

The method of preparing the above astringent formulation preferably comprises the steps of: mixing a double sulfate of a trivalent metal in combination with a univalent metal, cornstarch, water and a fragrance, wherein the double sulfate of a trivalent metal is one of: aluminum, chromium and iron and the univalent metal is one of: potassium and sodium. Preferably, as described above, the double sulfate of a trivalent metal in combination with a univalent metal is aluminum potassium sulfate. Preferably, the aluminum potassium sulfate comprises between 60% and 70% of the mixture, the cornstarch comprises between 10% and 20% of the mixture, and the water and fragrance comprise the balance of the mixture. More particularly, the aluminum potassium sulfate, cornstarch, and water are preferably in the approximate proportion of 65%, 15% and 20% respectively.

The method of using the above described astringent preferably comprises the steps of: applying the mixture of aluminum potassium sulfate as between 60% and 70% of the mixture, cornstarch as between 10% and 20% of the mixture, and the water and fragrance as the balance of the mixture to a skin surface, such as the face after shaving, and rinsing the mixture off the skin surface with water. A portion of the fragrance remains on the face after rinsing. It has been found that the formulation described has a novel effect in stopping minor bleeding very quickly while being thereafter able to be washed off the skin. The active ingredients; aluminum potassium sulfate and cornstarch n the stated proportions of the total formulation have been found to be critical in producing results in meeting the objectives of the present invention. It is the combination of these two ingredients that provides a significant and novel ability to stop bleeding instantly and sooth the face, while maintaining this action even if washed away immediately. After significant comparison with almost every known commercial product no equivalent has been found.

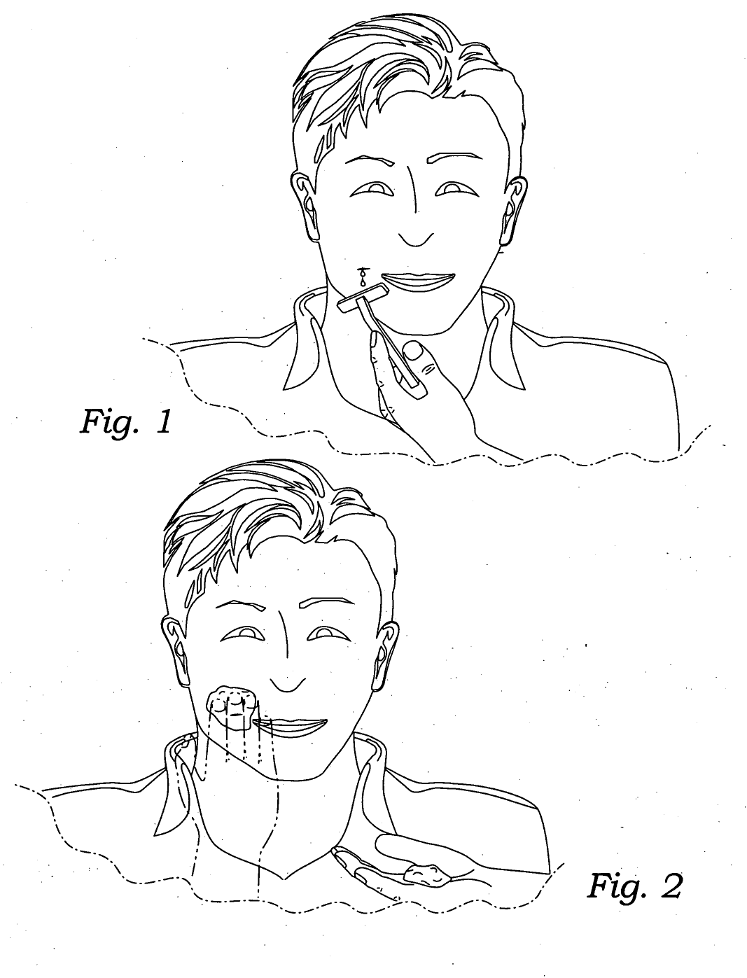

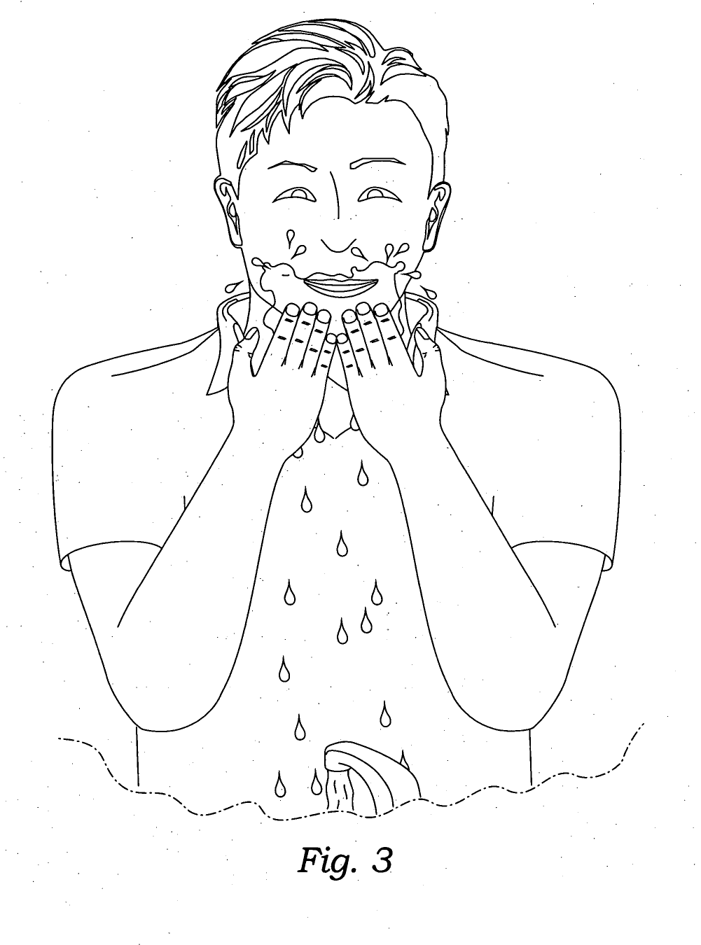

In the figures shown above, FIG. 1 shows an individual shaving and cutting himself; FIG. 2 shows application of the instant formulation; and FIG. 3 shows the same individual rinsing his face with water.

The enablements described in detail above are considered novel over the prior art of record and are considered critical to the operation of at least one aspect of the apparatus and its method of use and to the achievement of the above described objectives. The words used in this specification to describe the instant embodiments are to be understood not only in the sense of their commonly defined meanings, but to include by special definition in this specification: structure, material or acts beyond the scope of the commonly defined meanings. Thus if an element can be understood in the context of this specification as including more than one meaning, then its use must be understood as being generic to all possible meanings supported by the specification and by the word or words describing the element.

The definitions of the words or drawing elements described herein are meant to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described above, what is conceptually equivalent, what can be obviously substituted, and also what incorporates the essential ideas.

The scope of this description is to be interpreted only in conjunction with the appended claims and it is made clear, here, that each named inventor believes that the claimed subject matter is what is intended to be patented.

What is claimed is:

1. An astringent formulation comprising a mixture of: a double sulfate of a trivalent metal in combination with a univalent metal; cornstarch; water and a fragrance.

2. The mixture of claim 1 wherein the double sulfate of a trivalent metal is one of: aluminum, chromium and iron.

3. The mixture of claim 1 wherein the univalent metal is one of: potassium and sodium.

4. The mixture of claim 2 wherein the univalent metal is one of: potassium and sodium.

5. The mixture of claim 1 wherein the double sulfate of a trivalent metal in combination with a univalent metal is aluminum potassium sulfate.

6. The mixture of claim 5 wherein the aluminum potassium sulfate, cornstarch, and water are in the approximate proportion of 65%, 15% and 20% respectively.

7. The mixture of claim 5 wherein the aluminum potassium sulfate comprises between 60% and 70% of the mixture, the cornstarch comprises between 10% and 20% of the mixture, and the water and fragrance comprise the balance of the mixture.

8. A method of preparing an astringent formulation comprising the steps of: mixing a double sulfate of a trivalent metal in combination with a univalent metal, cornstarchc water and a fragrance.

9. The method of claim 8 wherein the double sulfate of a trivalent metal is one of: aluminum, chromium and iron.

10. The method of claim 8 wherein the univalent metal is one of: potassium and sodium.

11. The method of claim 9 wherein the univalent metal is one of: potassium and sodium.

12. The method of claim 8 wherein the double sulfate of a trivalent metal in combination with a univalent metal is aluminum potassium sulfate.

13. The method of claim 12 wherein the aluminum potassium sulfate, cornstarch, and water are in the approximate proportion of 65%, 15% and 20% respectively.

14. The method of claim 12 wherein the aluminum potassium sulfate comprises between 60% and 70% of the mixture, the cornstarch comprises between 10% and 20% of the mixture, and the water and fragrance comprise the balance of the mixture.

15. A method of using an astringent comprising the steps of: applying a mixture of aluminum potassium sulfate as between 60% and 70% of the mixture, cornstarch as between 10% and 20% of the mixture, and the water and fragrance as the balance of the mixture to a skin surface after shaving; and rinsing the mixture off the skin surface with water.
